(19) United States
(12) Patent Application Publication
(10) Pub. No.: US 2007/0087309 A1
(43) Pub. Date: Apr. 19, 2007

(54) Identification ring having an associated retaining clip, cover and stand

(76) Inventor: James Michael Campion, Buffalo, MN (US)

Correspondence Address: David George Johnson, Post Office Box 286, Aitkin, MN, 56431, US

(21) Appl. No.: 11/253,911

(22) Filed: Oct. 18, 2005

(51) Int. Cl. A61C 3/00; A61B 5/117

(52) U.S. Cl. 433/141; 433/229

(57) ABSTRACT

A clip assembly (57) retains a ring structure (35) which contains a plurality of individual color coded rings (7). A hand held tool (3), such as a dental instrument having a shaft (6) may be marked with a colored ring (7) by inserting the shaft (6) through the aperture (64) of the ring and separating the ring from the structure (35) by a twisting or pulling motion. The clip assembly (57) and the structure (35) are protected from contamination by transparent covers (65, 66). A stand (67) may be used to retain the clip assembly prior to and after use, with or without the presence of the covers (65. 66). The stand can also retain a tape dispenser (68) which contains colored tape (31) as may be needed for marking instruments that are not suitable for labeling with a ring (7).

### FIELD OF THE INVENTION

The present invention relates generally to the field dental instruments, and more particularly to a system for marking dental instruments by means of color codes.

### BACKGROUND OF THE INVENTION

While performing a typical dental procedure, a dentist or her assistant often utilizes a number of hand held instruments or tools. As dental technology has progressed, the number of such instruments has increased. A given procedure may require an array of instruments varying in shape, length and diameter. A common practice is to collect those instruments pertaining to a specific procedure and place them in a tray dedicated to that particular use. The variety of instruments, trays and dentists present in a dental office has prompted the use of color coding of both trays and instruments to correspond to a given procedure and for use by a specific practitioner. One method commonly used to identify a particular dental instrument is to form a colored ring, sleeve or disc on the handle of the instrument. An example of a dental instrument color coding scheme is disclosed in U.S. Pat. No. 4,882,867, entitled DENTAL INSTRUMENT HAVING IDENTIFICATION MARKINGS, issued on Nov. 28, 1989 to Linden. The Linden method is to permanently mold the colored sleeve, ring or disc into the handle, thereby enhancing the durability of the markings. The use of such a molded component is not entirely practical insofar as the color coded region may be raised with respect to the remainder of the handle surface, thereby interfering with the normal manipulation of the handle. Further, the molding process necessarily requires that the instrument handle be formed of a composite material which is inherently unsuited for repeated use in a sterilized environment.

In a sterilized environment where the dental tools have metallic handles, a different solution must be found. When using scalers and curettes, for example, the manufacturer typically applies a number to the handle of the instrument indicating the appropriate region of use. There is a conventional numbering system known as the Gracey System in which low numbers (less than five) designate anterior teeth and high numbers (greater than ten) designate the posterior bicuspid and molars. Since the numbers are stamped or etched directly into the handle they are necessarily small and have poor contrast. An effort to address this problem is disclosed in U.S. Pat. No. 4,988,295, entitled IDENTIFICATION MARKING SYSTEM FOR DENTAL INSTRUMENTS, issued on Jan. 29, 1991 to Kline. The instruments proposed by Kline contain a series of annular rings or grooves formed near one end of the handle, the number of rings and spacing indicating the area of use and general size of teeth or roots for which the given instrument has been manufactured. While the rings are certainly more visible than a stamped numeral, they are still relatively small marks which lack a contrasting color.

An effort to combine the advantages of a color coded handle with the durability of a stainless steel instrument is disclosed in U.S. Pat. No. 5,816,806, entitled DENTAL INSTRUMENTS WITH LARGE MOLDED HANDLES, issued on Oct. 6, 1998 to Herbst et al. The Herbst et al. device requires that the color coded indicator be integrally formed with the handle during the molding process.

Endodontic instruments are standardized by the International Standard Organization (ISO) Standard 3630, which defines the shape, length, size and handle color. Endodontic files and reamers can have sizes of between “10” and “150”, corresponding to standardized diameters. However, only six handle colors are utilized in the ISO identification scheme which is intended to apply to at least twenty different file sizes. This abbreviated color scheme requires the periodic repetition of the handle color, sometimes leading to confusion. In an effort to more precisely identify each endodontic file, a solution is proposed in U.S. Pat. No. 5,498,158, entitled COLOR-CODED ENDODONTIC CUTTING INSTRUMENTS AND COLOR-CODE SYSTEM AND METHOD, issued on Mar. 12, 1996 to Wong. The Wong device utilizes a two color scheme in which the grasping end of the file is integrally formed to include a first region having the first standardized color corresponding to the first smaller standard size and a second region having the second standardized color corresponding to the second larger standard size.

In an effort to avoid the limitation imposed by an integrally formed color coded handle, a removable ring is proposed in U.S. Pat. No. 6,193,515, entitled CODED DENTAL HANDLE, issued on Feb. 27, 2001 to Rahman. The Rahman device uses a color coded elastomeric ring that is located adjacent to a receiving port for a tool insert or point.

The Rahman device, as well as the schemes disclosed in the previously cited patents share a common construction characteristic insofar as the color coded portion or device cannot be relocated by the end user along the length of the instrument handle. Second, the color coding mechanism is not quickly and easily removed by hand should a change in the color coding change be desired. Third, the identification scheme is not universally applicable to a wide variety of existing dental instruments regardless of manufacturer. Fourth, the identifier is limited to the diameter of the handle itself, thereby limiting visibility. Fifth, the use of multiple identifiers at handle locations infinitely selectable by the end user is not possible.

Additional problems encountered in a real world dental practice include the cost of special identification schemes, incompatibility between systems obtained from different manufacturers, a lack of compatibility with other marking schemes such as colored tape and a lack of a sufficiently large number of available colors to create a unique color coding scheme for each possible scenario of unique types of procedures, unique steps to be performed as part of a given procedure, and a multiplicity of unique end users within a single dental office.

### SUMMARY OF THE INVENTION

The present invention is a color coded marking system for use in a dental office or similar setting where a plurality of different hand tools having dedicated uses and users are present. The present system includes a color coded ring that may be attached to the cylindrical handle of a dental instrument. The color coded ring is liquid injected molded with a silicone material. The rings are formed in interconnected batches from which an individual ring or rings may be removed as needed.

The molded rings are housed in a clip or dispenser having male and female halves which surround each batch of clips and protects the individual rings from accidental separation from the batch. A pair of covers may also be attached to the dispenser to provide further protection from accidental ring removal and to provide a relatively cleaner storage environment. The clip permits the marking of instruments without the need to directly handle the coded ring. Rather, the instrument may be inserted through the coded ring while still in the clip, thereby greatly reducing the possibility that the person applying the coded ring will be cut or impaled by the instrument.

The present invention also includes a dispenser stand or dispenser holder which is also adapted to housing colored tapes. The colored tapes are contained in individual dispensers and are used to mark instruments or items which are not readily adapted to receiving a code ring. The dispenser stand facilitates identifying and retrieving the code rings and tape dispensers in a manner that permits rapid identification of the colors available while maintaining stand portability and safety.

The molded color coded rings may be readily removed by an end user of the instrument in order to avoid interference with the use and manipulation of the instrument. The coded rings are contoured, durable, and pliable, and may safely continue to reside on the instrument during the cleaning and sterilization process.

### BRIEF DESCRIPTION OF THE DRAWINGS

FIG. 1 is a pictorial view of a code ring dispenser constructed according to the principles of the present invention;

FIG. 2 is pictorial view of a code ring assembly removed from the dispenser shown in FIG. 1;

FIG. 3 is a side view of the code ring assembly depicted in FIG. 2;

FIG. 4 is a sectional view of the code ring assembly taken along line 4-4 as shown in FIG. 3;

FIG. 5 is a sectional view of the code ring assembly taken along lines 5-5 as shown in FIG. 3;

FIG. 6 is a detail of the code ring assembly taken from region 43 as identified in FIG. 4;

FIG. 7 is a pictorial view of a code ring and tape dispenser stand constructed according to the principles of the present invention;

FIG. 8 is a truncated perspective view of the stand depicted in FIG. 7 showing the code ring dispenser of FIG. 1 housed therein;

FIG. 9 is an isometric view of the dispenser depicted in FIG. 7;

FIG. 10 is a plan view of the stand depicted in FIG. 7 shown with a code ring assembly, a code ring dispenser and a plurality of tape dispensers residing therein;

FIG. 11 is a sectional view taken along line 76-76 as shown in FIG. 10;

FIG. 12 is a side elevation view of the stand depicted in FIG. 10;

FIG. 13 is a rear elevation of the stand depicted in FIG. 12;

FIG. 14 is a detailed front elevation of the region 72 identified in FIG. 13;

FIG. 15 is a pictorial view of the dispenser depicted in FIG. 1 shown with both of the protective covers removed;

FIG. 16 is a pictorial view of the dispenser depicted in FIG. 1 shown with one of the protective covers removed;

FIG. 17 is a pictorial view of the code ring assembly mounted within the female half of the dispenser and without the male half of the dispenser shown in FIG. 1;

FIG. 18 is a pictorial view of dental instruments marked with color coded rings according to the principles of the present invention; and

FIG. 19 is a pictorial view of dental instruments marked with color coded tape according to the principles of the present invention.

### DETAILED DESCRIPTION OF THE INVENTION

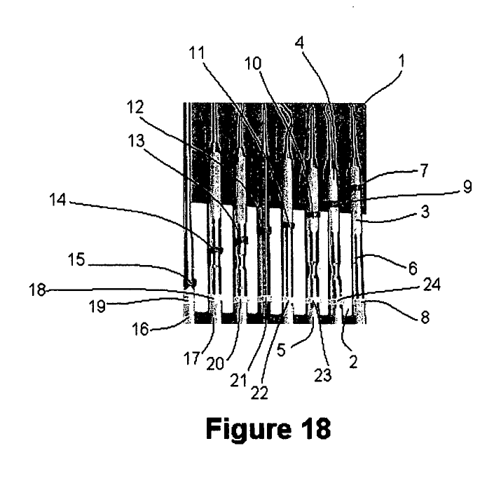

FIG. 18 illustrates a tray 1 which serves as a supporting surface for a dental tool or instrument holder 2. Residing within the holder 2 is a number of individual dental tools such as tools 3, 4 and 5, for example. Tool 3 is typical of many dental tools and has a shaft portion 6 that is intended to be grasped by a dental practitioner while using the instrument 3. Tool 3 is marked with a first code ring 7 and a second code ring 8. The first code ring 7 has a counterpart ring 9, 10, 11, 12, 13, 14 and 15 appearing on each of the remaining instruments residing in the holder 2. The first code ring 7 is of a first color which can be used to distinguish some characteristic of the tool, such as the particular procedure for which the tool is to be used. The rings 7 and 9-15 are arranged in unique positions along the shaft of each dental tool signifying the order in which each tool is to be used when performing the procedure. In the present example, the instrument 16 is intended to be used first, the tool 17 is to be used second and the tool 3 is intended to be used last while performing the procedure. A second set of individual code rings 8, 18, 19, 20, 21, 22, 23 and 24 are also affixed to the shaft of the dental instruments. The code rings 8 and 18-24 are of a second color and are used to signify a second characteristic of the instruments, such as the individual practitioner who is designated to utilize this particular set of tools.

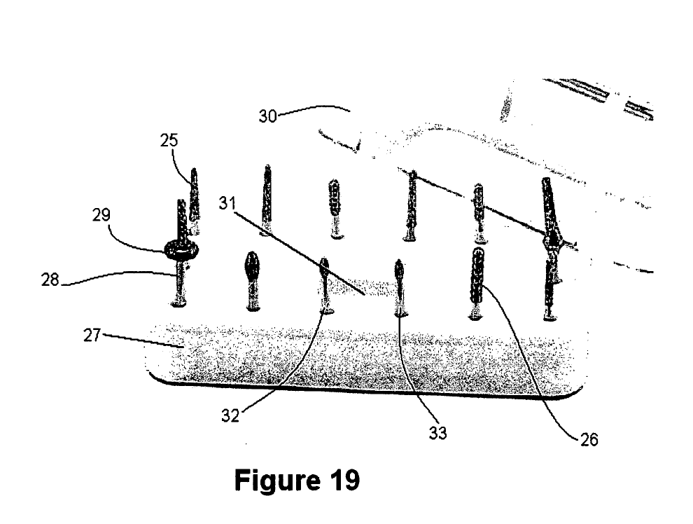

In some situations the use of a code ring 7 is not practical due to the size or shape of the dental tool. As depicted in FIG. 19, some dental tools such as files 25 and reamers 26. The tools 25 and 26 are shown housed in a tray 27 which partially conceals the shaft 28 of each instrument 29. A transparent cover 30 can be used to protect the instruments from dust while housed in the tray 27. The shafts 28 are too small or too obstructed to permit the use of a ring 7, and so a piece of tape 31 is used to either mark or even join adjacent instruments 32 and 33 which are to be used for a particular procedure or by a specific practitioner.

The placing of the code rings 7 or tape 31 on dental instruments is inherently problematical. The sterile end 34 of a typical dental instrument is often dangerously sharp, pointed or abrasive and in any event must be kept clean even if the tip 34 is relatively blunt or dull. Placing an individual ring 7 on an instrument shaft 6 invites the opportunity to puncture or prick the hand or finger of the person attempting to place and position the ring 7, and increases the probability that the hand will contaminate the sterile portion 34 the tool.

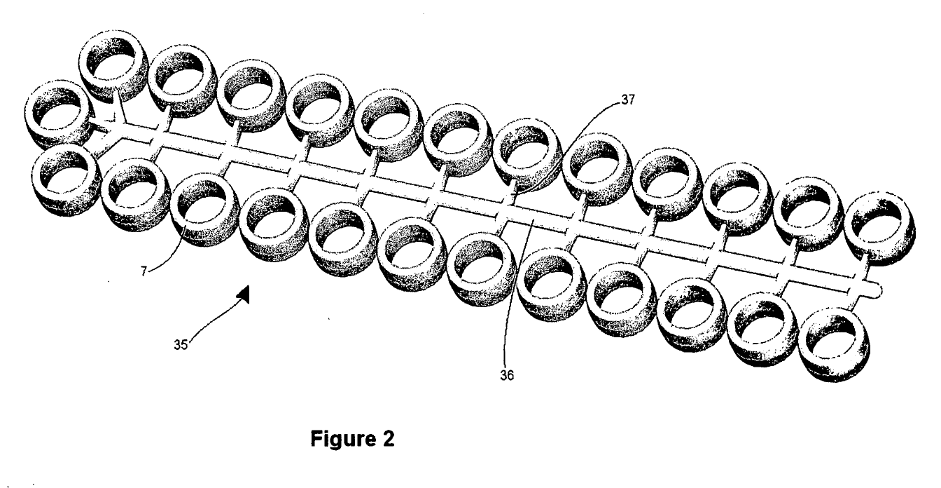

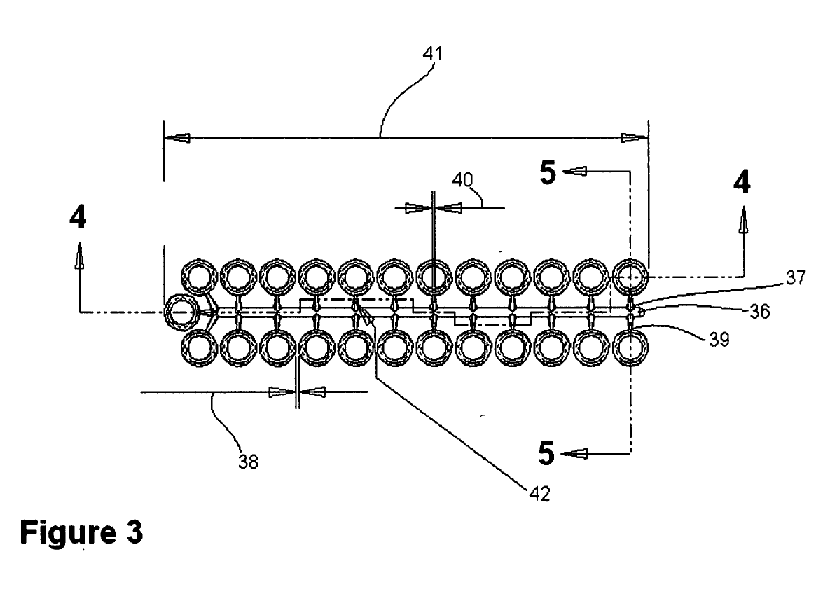

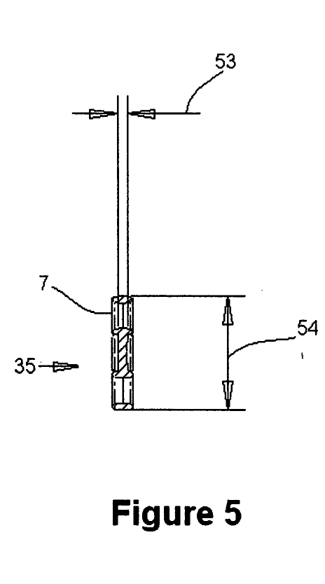

In order to increase the safety and simplicity of applying a ring 7 to a shaft 6, the rings are formed integrally as a set of multiple rings as shown in FIG. 2. Each ring 7 is formed by liquid injection molding silicone rubber to produce a structure 35 that includes twenty five individual rings 7 each joined to a central trunk 36 by means of branches 37. Referring also to FIG. 3, the characteristics of the structure 35 are important in permitting a user to readily remove a ring 7 for use. Since the present invention may be used to accommodate differing tool shaft diameters and shapes, the ring 7 is preferably manufactured in multiple sizes. In the case of a relatively small ring 7, the dimension 38 is approximately 0.051 inch, while for a relatively large ring the dimension 38 is approximately 0.021 inch. Each branch 37 tapers to a break point 39 at which the ring 7 may be readily separated from the branch 37. The dimension 40 corresponding to the longitudinal width of the break point 39 is approximately 0.020 inch for the smaller ring 7 and is approximately 0.019 inch for the larger ring 7. As best seen in FIG. 5, the lateral width 53 of the break point 39 is approximately 0.63 inch. The attachment point 42 which joins the branch 37 to the trunk 36 is necessarily larger in cross section than the diameter 40 in order to insure that the ring 7 separates from the structure 35 at the break point 39 when a force is applied to the ring 7. The overall length 41 of the structure 35 is approximately 3.446 inches for the smaller ring 7 and is approximately 3.476 inches for the larger ring 7. The overall height 54 of structure 35 is approximately 0.730 inch for the smaller ring 7 and 0.760 inch for the larger ring 7.

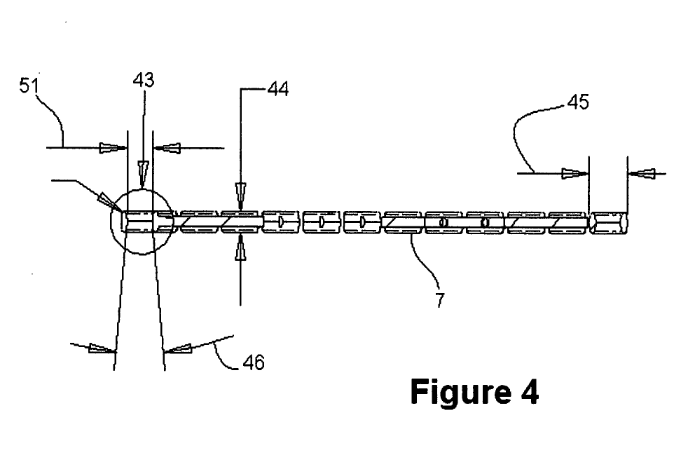

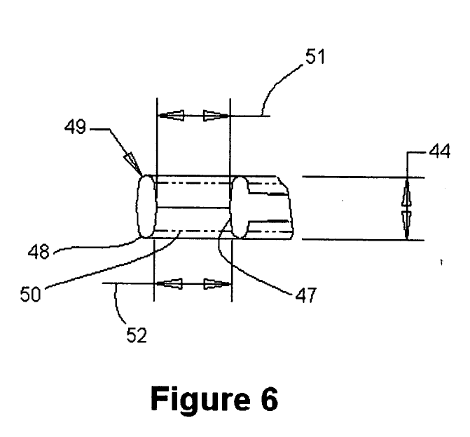

Referring also to FIG. 4, the height 44 of the ring 7 is 0.140 inch. The diameter 45 of the smaller ring 7 is 0.230 inch and the diameter 45 of the larger ring 7 is 0.260 inch. As best seen in FIG. 6, each ring 7 has a beveled or tapered interior surface 47. The angle 46 of the bevel 47 is approximately ten degrees. Further, the ring 7 has a curved edge 48 having a radius 49 of 0.017 inch. The curved edge 48 joins the beveled surface 47 at line 50. The smallest inner diameter 51 has a value of 0.140 inch for the smaller ring 7 and a value of 0.170 inch for the larger ring 7. The diameter 52 of the inscribed circle formed by the line 50 is equal to 0.150 inch for the smaller ring 7 and 0.180 inch for the larger ring 7. The tapered interior surface 47 causes the ring 7 to present a varying inside diameter to the instrument shaft 6, simplifying insertion of the ring onto the shaft. The ring 7 is composed of a resilient silicon rubber material that deforms sufficiently to cause substantially all of the interior surface 47 to abut the instrument shaft 6 once the ring assumes a final mounting position on the shaft.

The structure 35 is composed of a silicone rubber material and is relative pliable. In other words, the trunk 36 and branches 37 lack sufficient stiffness to maintain the shape shown in FIG. 2 without substantial deformation. In order to permit the rings 7 to be safely and accurately applied to a dental instrument the structure 35 must be externally supported.

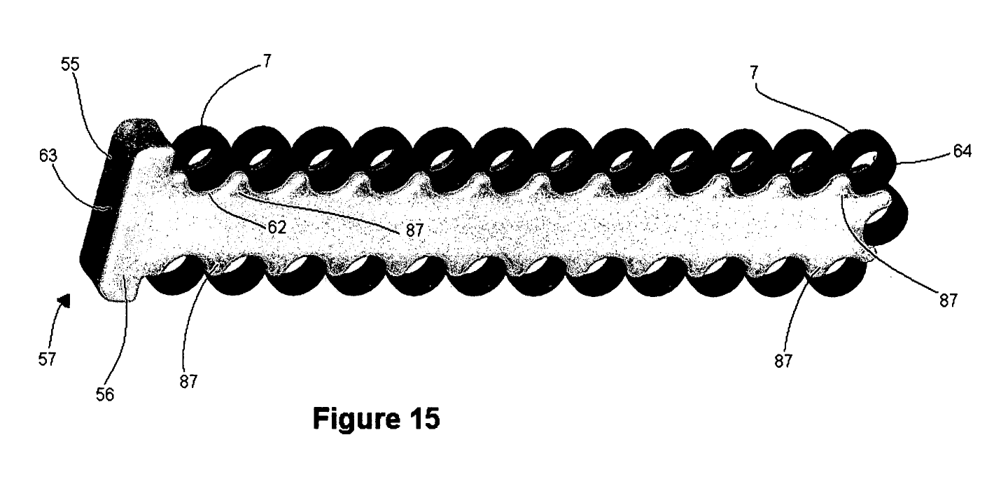

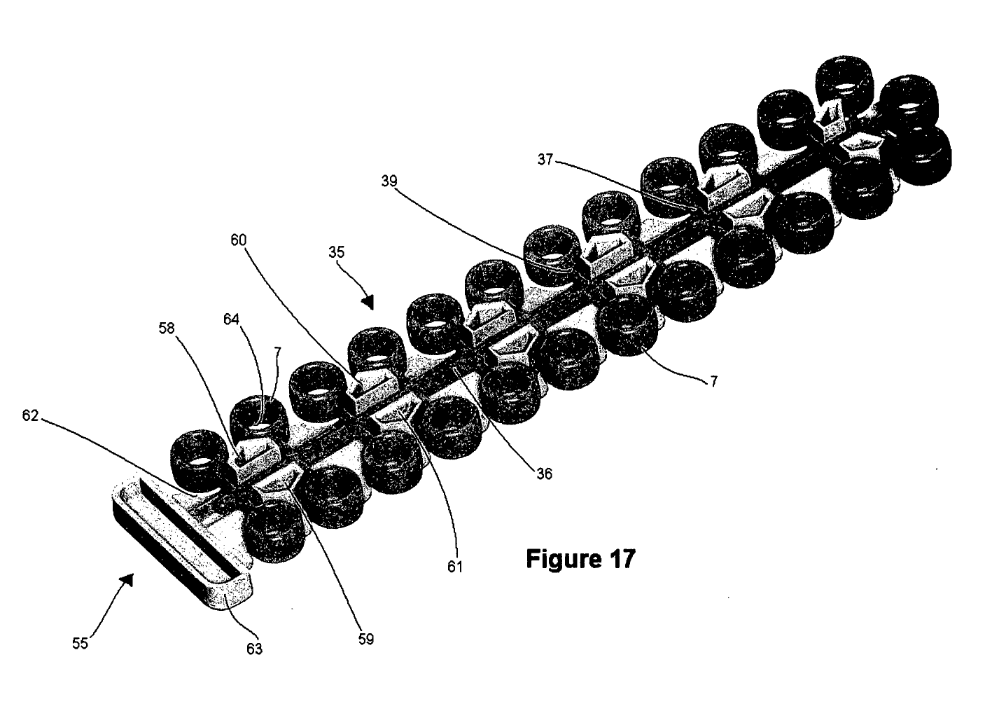

Referring to FIG. 17, the structure 35 is shown residing in a first clip 55. Referring also to FIG. 15, the first clip 55 is seen to be the female half which is adapted to join a second clip 56 to form a complete clip assembly 57. A series of sockets 58, 59, 60 and 61, for example, are formed in the first clip 55 and are adapted to receive an aligned pin, wedge or other fastener formed within the second clip 56. The completed clip assembly 57 creates a series of scalloped edges 62 which expose each of the rings 7 of the structure 35. Each clip assembly half 55 and 56 includes a handle or grip portion 63 that is suitably dimensioned to permit grasping the handle 63 between, for example, a thumb and index finger. When a practitioner desires to place a ring 7 on a dental instrument shaft, the practitioner grasps the clip handle 63 with one hand and, using the other hand, inserts the dental instrument shaft through the aperture 64 formed by each ring 7. By applying a slight relative twisting and pulling motion between the shaft 6 of the dental instrument and the clip assembly 57, the ring 7 will separate from the structure 35 at the break point 39. The ring 7 is thus securely surrounding the shaft 6 and may then be safely repositioned on the shaft, if necessary, by finger manipulation.

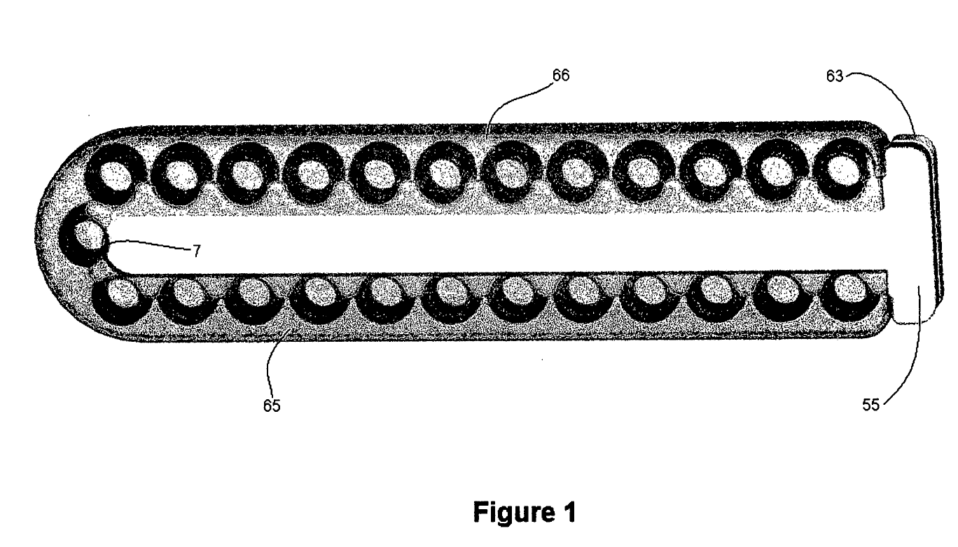

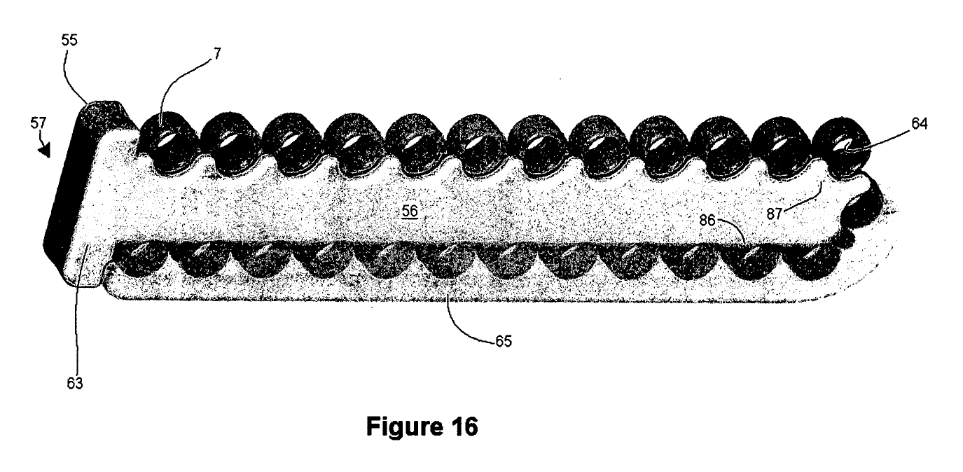

In order to preserve the sanitary condition of each ring 7 prior to use some form of protection must be provided. Referring to FIG. 16, the clip assembly 57 is provided with a removable first cover 65. The cover 65 is preferably composed of a transparent material which permits a user to determine the color of the rings 7, the quantity of rings available within the clip assembly 57 and the location of each remaining ring. As shown in FIG. 1, a second transparent cover 66 encloses the remaining exposed portion of the structure 35. The covers 65 and 66 are substantially identical and interchangeable. The covers 65 and 66 are composed of a material that is capable of slight deformation, thereby permitting the edges 86 of each cover to abut and grip a series of bosses 87 extending from the clip assembly 57.

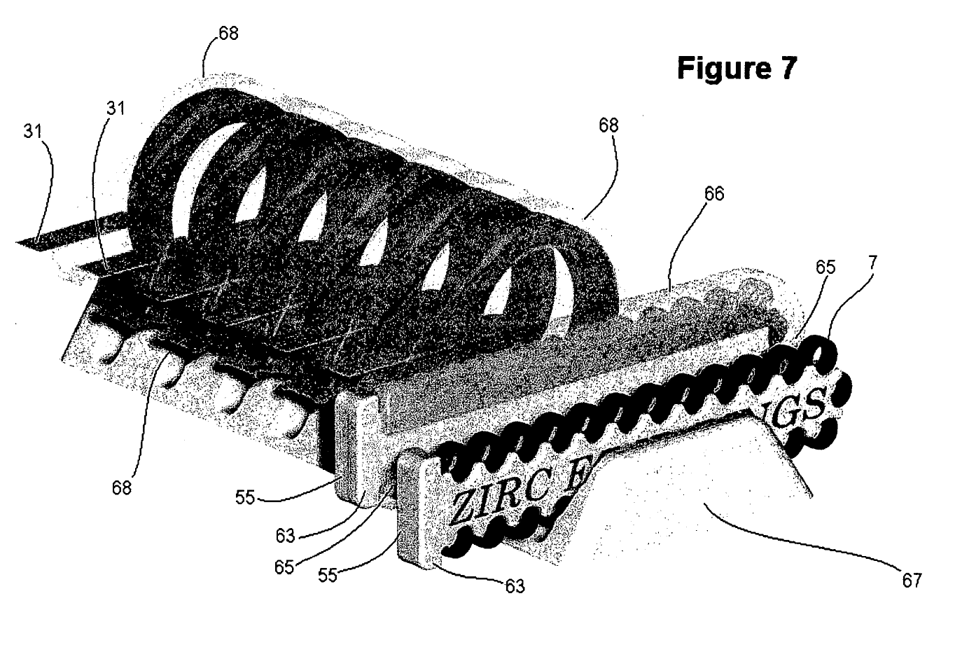

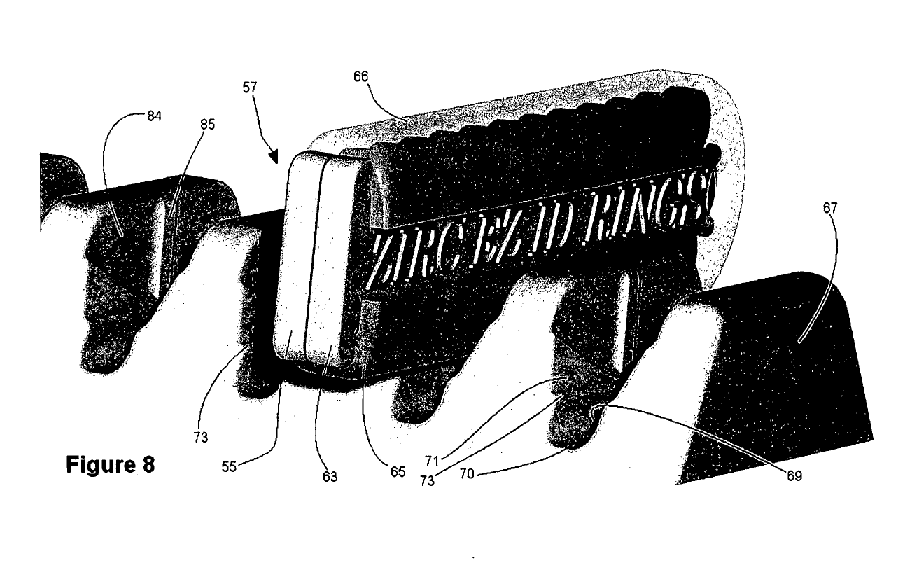

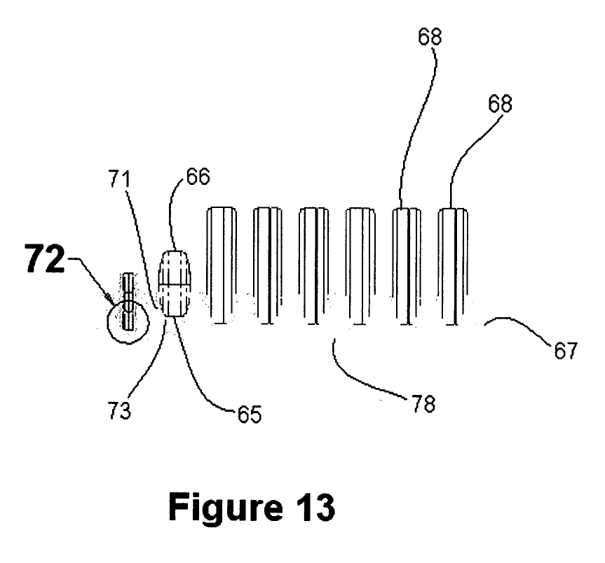

In the operatory of a typical dental office, some means must be provided for conveniently storing and providing access to the clip assembly 57. Referring to FIG. 7, a stand 67 is shown that is adapted to house the clip assembly 57 without the cover 65 installed and with the cover 65 installed. Further, the tape 31 is housed in transparent dispensers 68 which may also be securely placed within the stand 67. Referring also to FIG. 8, the clip assembly 57 complete with both covers 65 and 66 in place is shown housed in the stand 67. A series of bosses or ledges are formed within the sides of the clip retaining groove 69 in order to securely accommodate whatever item may be placed in the groove. The beveled wall 71 is adapted to intercept and retain the exterior surface of lower cover 65. As best seen in FIG. 13, the stand 67 is slightly deformable and sufficiently resilient to grip the lower cover 65 as the covered assembly 57 is place within groove 69.

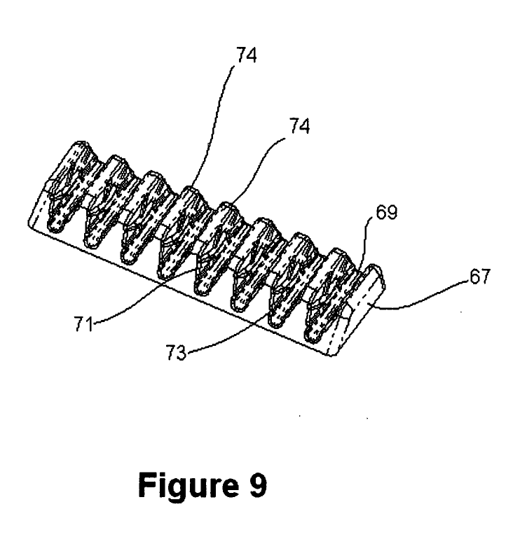

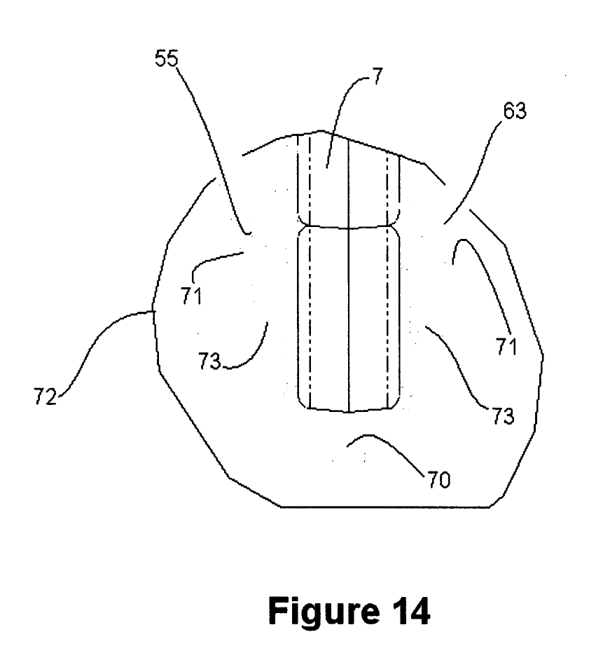

In the case of the assembly 57 with the bottom cover 65 removed, the rings 7 rest slightly above the floor 70 of the groove 69 as depicted in FIG. 14. The clips 55 and 63 are retained by the boss 73 formed at the base of the beveled wall 71. Referring also to FIG. 9, each groove 69 is separated from the adjacent groove by a divider 74.

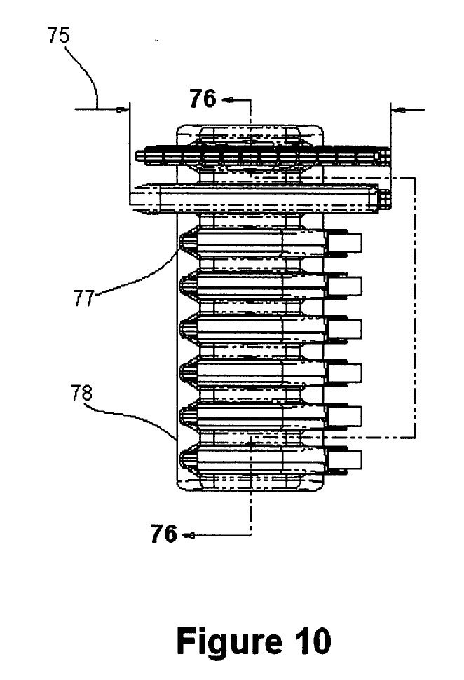

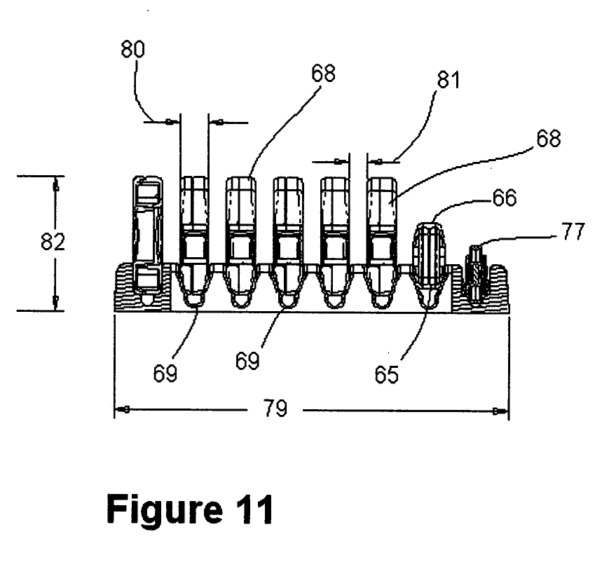

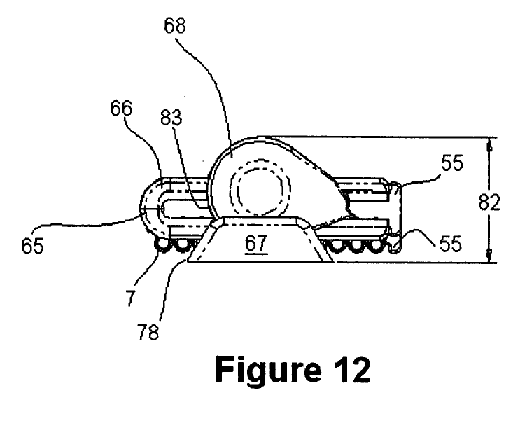

The stand 67 is also adapted to house the tape dispenser 68. As seen in FIG. 10, a typical tape dispenser 68 has a depth that is somewhat less than the depth 75 of a clip assembly 57, complete with the top cover 66 and bottom cover 65 in place, which is approximately 3.814 inches. The rear edge 77 of the tape dispense 68 extends almost to the rear bottom edge 78 of the stand 67. Referring also to FIG. 11, the width 80 of each tape dispenser 68 is approximately 0.3820 inch and the height 82 of each dispenser is approximately 1.7778 inches. The width 81 of each divider 74 is approximately 0.2430 inch. The overall width 79 of a typical stand 67 is approximately 5.25 inches which provides for eight grooves 69 for accommodating various articles. As best viewed in FIG. 12, the tape dispenser 68 has a curved perimeter 83 which mates with the indentation 84 visible in FIG. 8. The boss 85 extending from the indentation 84 tends to grip and secure the dispenser 68 within the groove 69.

From the foregoing one can readily foresee that numerous modifications to the foregoing may be made without departing from the spirit and the scope of the invention. For example, the rings 7 may be any type of fastener or label is capable of being impaled, snagged or gripped by an instrument or tool which is intended to be marked for identification. The clip assembly 57 may be modified as desired to either surround or secure the fastener or label formed on the structure 35 so that the fastener or label is accessible. The stand 67 may be modified as desired to support various embodiments of the clip assembly 57 or other tape dispensers 68. Further, the dimensions of the rings 7 and the clip assembly 57 may be modified as necessary to accommodate a tool or instrument having a particular size or shape. The claims define the scope of the invention.

I claim:

1. An instrument shaft marking system including a ring structure, wherein the ring structure comprises;
a trunk;
at least one branch extending from the trunk; and
a ring, the ring being affixed to and formed integrally with the branch, the ring being suitably dimensioned to permit an instrument shaft to be inserted within the ring.

2. The instrument marking system of claim 1, further comprising a break point, the break point being formed as a region of reduced cross section residing at an intersection of the branch and the ring, thereby facilitating removal of the ring from the branch while leaving the ring substantially free of evidence of attachment to the branch.

3. The instrument marking system of claim 2, further comprising a clip assembly, the clip assembly abutting the trunk and each branch so as to retain each ring in an exposed position, thereby facilitating orientation of each ring when inserting the instrument shaft within the ring.

4. The instrument marking system of claim 3, further comprising at least one clip cover, the clip cover being affixed to the clip assembly so as to protect the ring prior to placement on an instrument.

5. The instrument marking system of claim 4, wherein the trunk comprises an elongated member composed of a flexible material, the trunk being formed integrally with a plurality of branches extending from the trunk, wherein each branch is formed of the flexible material, each branch being substantially coplanar with the trunk and all other branches extending from the trunk.

6. The system of claim 5, wherein each ring is formed integrally with the trunk, the ring being composed of the flexible material, each ring being formed to include a tapering interior surface which presents a varying inside diameter to an instrument shaft.

7. The instrument marking system of claim 6, wherein substantially all of the interior surface of the ring abuts the instrument shaft when the ring assumes a final mounting position on the instrument shaft.

8. The instrument marking system of claim 6, wherein each ring is removable from the branch that anchors the ring to the trunk by at applying at least one of a) a torsional force and b) a pulling force, to the ring with respect to the branch.

9. The instrument marking system of claim 7, further comprising a pair of substantially transparent clip covers, each cover being substantially identical, each cover being adapted to cover approximately one half the rings extending from the clip assembly.

10. An instrument marking system stand for organizing and providing access to at least one instrument marking accessory, comprising:
a base; and
a plurality of spaced apart grooves, each groove being adapted to retain an individual instrument marking accessory.

11. The instrument marking system stand of claim 10, wherein the individual instrument marking accessory is one of a group comprising:
a tape dispenser;
a completely covered ring retaining clip assembly;
a partially covered ring retaining clip assembly; and
an uncovered ring retaining clip assembly.

12. The instrument marking system stand of claim 11, further comprising a series of ledges formed within each groove, each ledge being shaped and dimensioned to securely accommodate any of the group of including each individual marking accessory that is placed in the groove.

13. The instrument marking system stand of claim 11, wherein at least one ledge is formed as a beveled wall adapted to intercept and retain an exterior surface of a clip assembly cover.

14. The instrument marking system stand of claim 13, wherein the stand is formed of a relatively deformable material having sufficient resilience to grip the exterior cover of a clip assembly placed within the groove.

15. The instrument marking system stand of claim 14, wherein at least one ledge further comprises a boss adapted to intercept and retain a tape dispenser residing within the groove.

16. The instrument marking system stand of claim 15, wherein each groove further comprises:
a floor; and
a clip retaining boss formed within the beveled wall 71, the clip retaining boss being adapted to intercept and retain a portion of the partially covered and the uncovered clip assembly such that a ring residing within the clip assembly is supported in a spaced apart relationship from the floor.

17. The instrument marking system stand of claim 16 wherein each groove is separated from an adjacent groove by a divider.

18. A method of marking a shaft of a dental instrument, comprising the steps of:
forming a ring at the end of a protruding branch;
inserting the shaft through the ring; and
twisting the ring relative to the protruding branch so as to separate the ring from the branch, thereby causing the ring to be affixed to the shaft.

19. The method of marking a shaft of claim 18, further comprising the steps of:
forming a plurality of protruding branches integrally with a central trunk, each branch extending from the trunk; and
forming an intersection between each branch and ring so as to have a reduced cross sectional area relative to the branch, thereby creating a breakage point at the intersection between the branch and the ring.

20. The method of marking a shaft of claim 19, further comprising the steps of:
forming a clip adapted to grip and retain the central trunk and each protruding branch, the clip being gripped by a user so as to safely position each ring relative to an instrument shaft; and
covering at least some rings retained within the clip with a transparent cover so as to protect the rings prior to use and to provide a ready indication of the rings remaining when the transparent cover is placed over the rings.
